(19) United States
(12) Patent Application Publication
(10) Pub. No.: US 2007/0191929 A1
(43) Pub. Date: Aug. 16, 2007

(54) Medical devices using magnetic pulse welding

(75) Inventors: Thomas A. Osborne, Bloomington, IN (US); Andrew J. Herald, Bloomington, IN (US); Scott E. Eells, Bloomington, IN (US)

Correspondence Address: BRINKS HOFER GILSON & LIONE, P.O. BOX 10395, CHICAGO, IL, 60610, US

(73) Assignee: Cook Incorporated

(21) Appl. No.: 10/922,567

(22) Filed: Nov. 22, 2004

Related U.S. Application Data
(60) Provisional application No. 60/498,016, filed on Aug. 27, 2003.

(51) Int. Cl. A61F 2/90

(52) U.S. Cl. 623/1.16; 623/1.36; 228/107

(57) ABSTRACT

Medical devices for use in or on a mammalian body have improved properties through the use of magnetic pulse welding to join components. The improved properties include increased strength, corrosion resistance, and preservation of material characteristics. A method of forming a medical device using magnetic pulse welding is also provided.

### PRIORITY CLAIM

This application claims priority to Provisional Application Ser. No. 60/498,016 which was filed on Aug. 27, 2003.

### BACKGROUND

This invention relates to medical devices with improved properties, and more particularly to methods of manufacturing medical devices with improved properties using magnetic pulse welding.

Many medical devices are currently assembled through the use of soldering, brazing, welding, and/or through the use of adhesive bonding. Some examples of typical devices that use these assembly methods include stent connections, filters, needle assemblies, wire guide assemblies, needle cannula assemblies, retrieval basket assemblies, snare loop assemblies, and spider occluder assemblies. However, this list is not exhaustive but rather exemplary. Some of these medical devices, for example, stents, filters, and spider occluders, remain in the patient indefinitely. Others, such as wire guides, and needles, are only in the patient for short periods of time.

The method used to assemble these devices is chosen based on several factors including but not limited to strength, corrosion resistance, and time in the patient. Soldering, for example, may be chosen for applications that require low temperature during assembly and when exposure time in the patient is limited. An exemplary device using soldering is a small wire guide where welding or brazing would damage the thin, small wires and create weak areas where the wire could break under moderate loads. Brazing or welding may be used when the assembly is sufficiently robust to withstand the high temperature of the processes. Adhesive bonding may be used when the device is small and fragile and will be exposed to the blood stream for extended periods of time.

However, each of these processes compromises one or more important device properties, such that it may decrease device strength, lessen corrosion resistance, degrade the material, decrease the springiness or flexibility of the material, increase the size of the manufactured device, lessen the smoothness of the material, or decrease the uniformity or precision of the results of the processes, and as a result yield less than ideal results. Additionally, these processes can require heating, and/or be time consuming and thereby increase manufacturing costs.

What has been needed and until present unavailable in the art of medical devices is a manufacturing process which meets the demanding requirements of many medical devices for maximum strength, corrosion resistance, and preservation of material characteristics. The present invention satisfies these and other needs.

### SUMMARY

It is in an object of the invention to improve the method of manufacturing a medical device for use in or on a mammalian body.

In one aspect, a medical device for use in or on a mammalian body comprises a first component, a second component, and a connection formed between the first component and the second component. The connection is formed by magnetic pulse welding.

In another aspect, a medical device for use in or on a mammalian body comprises a first conductive, metallic component and a second conductive, metallic component. At least a portion of the first component and at least a portion of the second component are joined in a metallurgical bond without degradation of the metal of either component.

In yet another aspect, a method of assembling at least a portion of a medical device for use in or on a mammalian body is provided. A first component, a second component, and an inductor are provided, wherein at least one of the first and second components is conductive. At least a portion of the first component is positioned near at least a portion of the second component in the vicinity of the inductor. A current pulse is supplied to the inductor to generate a magnetic field which causes at least a portion of the first component to form a connection with at least a portion of the second component through magnetic pulse welding.

In another aspect, a method of assembling at least a portion of a medical device for use in or on a mammalian body is provided. A first component, a second component, and an inductor are provided, wherein at least one of the first and second components is conductive. At least a portion of the first component is positioned near at least a portion of the second component in the vicinity of the inductor. One of the first and second components is explosively compelled towards the other of the components through the use of a magnetic field.

In a final aspect, a method of assembling at least a portion of a medical device for use in or on a mammalian body is provided. A first component made of a material, a second component made of a material, and an inductor are provided, wherein at least one of the first and second components is conductive. At least a portion of the first component is positioned near at least a portion of the second component in the vicinity of the inductor. One of the first and second components is explosively compelled towards the other of the components without substantially heating or degrading the material of either of said first and second components.

For purposes of the invention, the components of the medical device may be tubular. Both of the components may be conductive, or one of the components may be conductive and the other component non-conductive. The magnetic pulse welding process may take less than 100 microseconds. The components may be heated to less than about thirty degrees Celsius during the welding process, and if the components are metallic they may be joined in a metallurgical bond without degradation of the material of either component.

Manufacturing a medical device utilizing a magnetic pulse welding process may increase strength and corrosion resistance, and preserve material characteristics.

The present invention, together with further objects and advantages, will be best understood by reference to the following detailed description taken in conjunction with the accompanying drawings.

### BRIEF DESCRIPTION OF SEVERAL VIEWS OF THE DRAWINGS

FIG. 1 is a side view of one embodiment of a stent device.

FIG. 2 is a cut open and unfolded view of a segment of interconnected spring members in a stent device.

FIG. 3 is an enlarged segment showing a connecting member fastened to an arm section in a spring member of a stent device.

FIG. 4 is a view of one half of a stent device shown without graft material.

FIGS. 5-7 are enlarged views of a connecting member and spring members in a stent device in an unloaded state, at an axial pressure load, and at an axial tensile load, respectively.

FIG. 8 is a cross-section diagrammatic view of one embodiment of a configuration for magnetic pulse welding.

FIG. 9 is a partial view of one embodiment of a configuration for magnetic pulse welding.

FIG. 10 is a partial view of one embodiment of a configuration for magnetic pulse welding.

FIG. 11 is a partial view of one embodiment of a configuration for magnetic pulse welding.

FIG. 12 is a partial view of one embodiment of a portion of a stent assembled according to the invention.

FIG. 13 is a partial view of another embodiment of a portion of a stent assembled according to the invention.

FIG. 14 is a partial view of another embodiment of a portion of a stent assembled according to the invention.

FIG. 15 is a partial view of one embodiment of a stent having anchoring barbs to which the invention could be advantageously applied.

FIG. 16 is a partial view of one embodiment of a stent having an anchoring barb under the prior art to which the invention could be advantageously applied.

FIG. 17 is a partial view of one embodiment of a stent having anchoring barbs assembled according to the invention.

FIG. 18 is a partial view of one embodiment of an abdominal aorta aneurysm (AAA) device to which the invention could be advantageously applied.

FIG. 19 is a partial view of one embodiment of a clot filter to which the invention could be advantageously applied.

FIG. 20 is a partial view of one embodiment of a cylinder type assembly to which the invention could be advantageously applied.

FIG. 21 is a partial view of one embodiment of a marker band over a catheter to which the invention could be advantageously applied.

FIG. 22 is a partial view of one embodiment of a wire guide assembly to which the invention could be advantageously applied.

FIG. 23 is a partial view of one embodiment of a tip deflecting wire to which the invention could be advantageously applied.

FIG. 24 is a partial view of one embodiment of an assembly of a needle cannula attached to a metal hub to which the invention could be advantageously applied.

FIG. 25 is a partial view of one embodiment of a retrieval basket to which the invention could be advantageously applied.

FIG. 26 is a partial view of one embodiment of a retrieval snare to which the invention could be advantageously applied.

FIG. 27 is a partial view of one embodiment of a spider occluder device to which the invention could be advantageously applied.

FIG. 28 is a partial view of one embodiment of a breast lesion localization device to which the invention could be advantageously applied.

FIG. 29 is a partial view of one embodiment of a needle to which the invention could be advantageously applied.

### DETAILED DESCRIPTION OF THE PREFERRED EMBODIMENTS

As shown in the drawings for purposes of illustration, the present invention is directed to the manufacture of medical devices with improved properties previously unknown in the art of medical devices. Virtually any medical device which is implanted or used in or on a mammalian body, such as a person, could benefit from the present invention. Examples of such medical devices include stent connections, filters, needle assemblies, wire guide assemblies, needle cannula assemblies, retrieval basket assemblies, snare loop assemblies, and spider occluder assemblies. However, this list is not exhaustive but rather exemplary.

In one embodiment, the present invention is utilized in a stent. Stents are well known in the art and the description herein of stents is for example only and is not meant to be limiting. The present invention is applicable to stents having different types of configurations and different deployment systems. The stent may be self-expanding or balloon expandable.

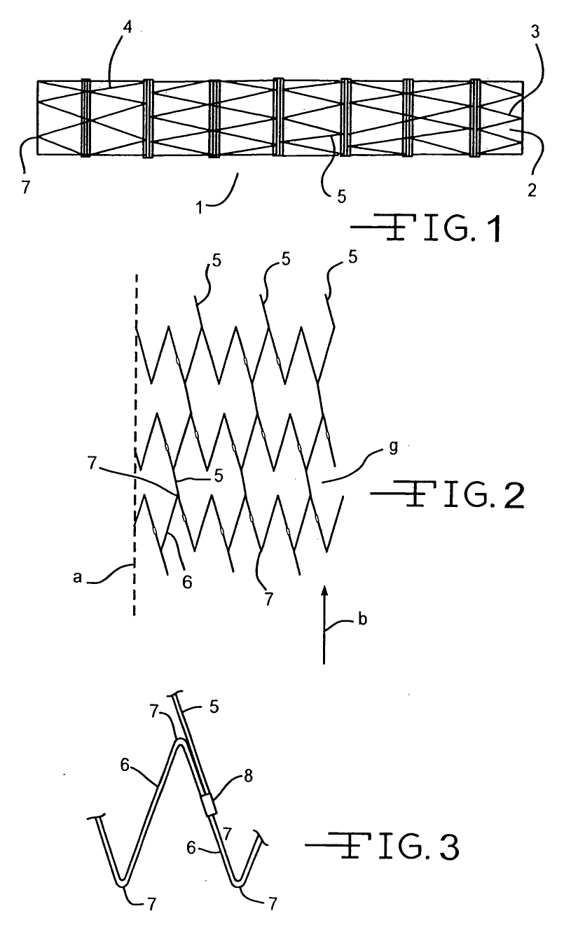

An example of a stent is disclosed in Australian patent number 729883 to William Cook Europe A/S which is hereby incorporated by reference. As shown in FIG. 1, a stent device 1 may be formed as a single tube, but a stent may also in other embodiments comprise a branched structure such as a Y-shaped structure with a relatively large tube branching into two smaller tubes. The stent device as a single tube can be used for treatment of a single-lumen vessel, such as to recreate the lumen of the vessel at a stenosis, for use as a vessel prosthesis at an aneurysm or a fistula, or at some other vessel anomaly. The stent device 1 comprises a graft 2 supported by a tubular member 3, which is constructed from a plurality of cylindrical spring members 4 interconnected by connecting members 5. A stent device with a Y-shaped structure may be used to treat an abdominal aorta aneurysm.

The individual spring members 4 are Z-stents of a well-known design made of a wire of stainless steel, nitinol, a resilient plastics material, or a metallic material or composite which can exhibit elastic or superelastic properties. The wire is formed in a zig-zag configuration extending through a cylindrical surface, and the wire ends are joined together to make it endless, so that the cylindrical spring member is closed. FIG. 2 shows three spring members which are cut open along the line a and unfolded to a planar shape for the sake of clarity. The longitudinal direction of the tubular member is indicated by the arrow b. It should be noted that the different figures use the same reference numerals for details of the same type.

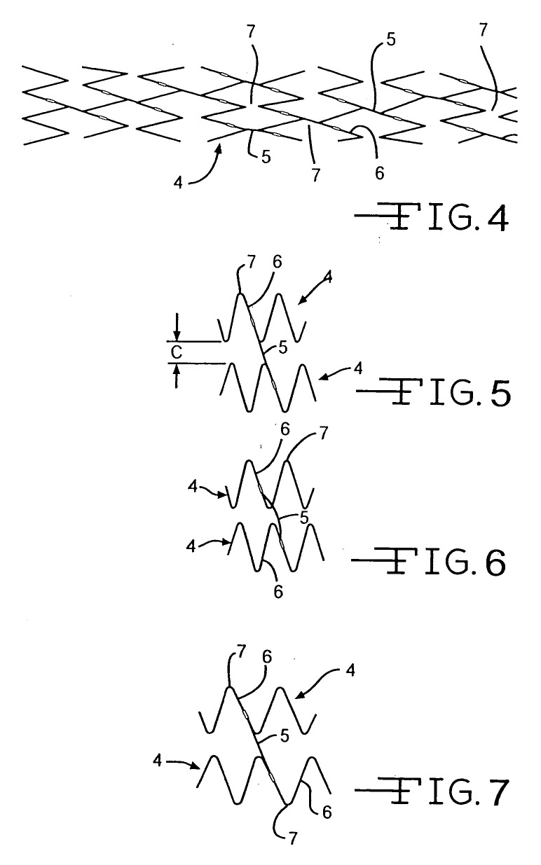

A spring member 4 includes a plurality of arm sections 6, each extending between two elbow sections 7. The arm sections are shown as approximately straight in their unloaded state, which is preferred to obtain high axial rigidity, but they may also be curved. The elbow sections are shown as simple bends in which the wire extends in a simple angle of a suitable radius of curvature. However, the elbow sections may have a more complex design in which the wire at the end of the arm section is formed, for example, as shown in FIG. 5 in EP-A1 0539237.

As shown in FIG. 2, the connecting members 5 may extend obliquely in relation to the longitudinal direction b of the stent device 1. In the stent's expanded and unexpended states, the connecting members are seen to extend largely in parallel with the arm sections 4 and also mutually in parallel. The connecting members 5 can also be made of a wire of stainless steel, nitinol, a resilient plastics material, or a metallic material or composite which can exhibit elastic or superelastic properties.

The embodiment shown in FIG. 2 is one preferred stent arrangement, although other arrangements may also be utilized. As shown in FIG. 2, a gap g follows a pair of interconnected arm sections, followed by another pair of interconnected arm sections. In the gap, the elbow sections are free to move closer to or further away from each other.

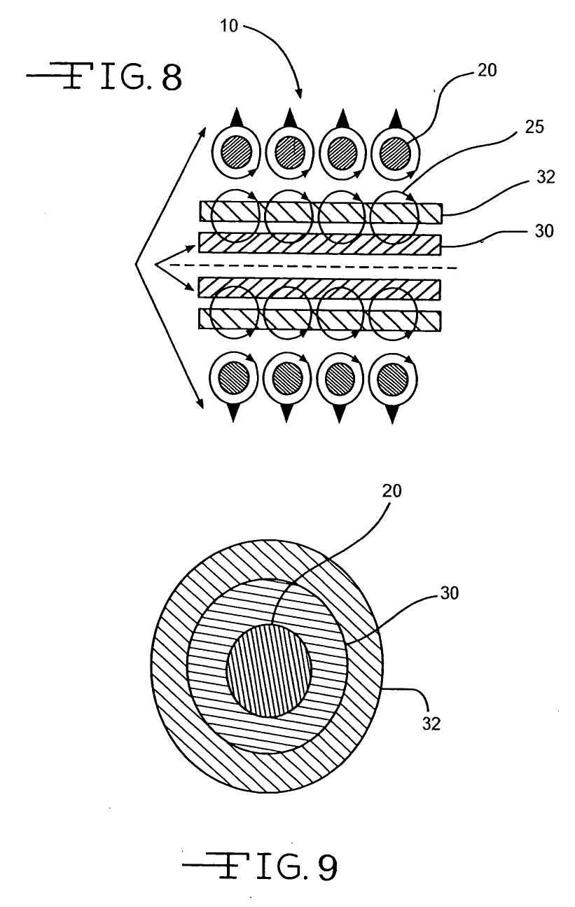

FIG. 4 shows an expanded view of the arrangement of FIG. 2. In the case of the advantageous locations of the connecting members 5 shown in FIG. 4, between each of the arm sections having a mounted connecting member 5 there is an arm section without any connecting member. Thus both in the circumferential direction and in the longitudinal direction there is a free elbow section. With this open structure, the individual spring members can be resiliently deformed as indicated in FIGS. 5-7. FIG. 5 shows the unloaded state in which the spring members 4 are maintained at the mutual distance c by means of the connection member 5. The distance c can be freely adapted to the specific application by choice of a suitable length of the members 5.

At their ends the connecting members 5 are firmly fastened to the associated arm sections 6. FIG. 3 shows that the fastening point 8 is at a distance from the elbow sections 7. Fastening is performed at both ends of the connecting member. The connection may also be made in other areas, such as a distance away from the end sections of the connecting member. Up until the present invention, the fastening was performed, e.g., using means of soldering, brazing, welding, adhesive bonding, and/or geometrical locking with mutual geometrical engagement between the connecting member and the arm section. However, each of these prior attachment methods has one or more deficiencies.

For instance, soldering is the process of making a joint between metals by joining them with a soft solder. This may be a low temperature melting point alloy of lead and tin. The joint is typically heated to the correct temperature, which is typically around 250 degrees Centigrade, by a soldering iron, at a temperature well below the melting point of the metals being connected. Soldering may be chosen for applications that require low temperature during assembly and when exposure time in the patient is limited. An exemplary device using soldering is a small wire guide where welding or brazing would damage the thin, small wires and create weak areas where the wire could break under moderate loads. However, a disadvantage of soldering is that the joint is not as strong as the metals themselves, the solder increases the size of the joint and may be non-uniform, the joint cannot be subjected to high temperatures, and the solder may break down and be absorbed into the body with potential negative health complications.

Brazing is the joining of metals through the use of heat and a filler metal whose melting temperature is typically above 450 degrees Centigrade but below the melting point of the metals being joined. Brazing gives the advantages of making a strong, permanent joint. However, a disadvantage of brazing is that the heat must be applied to a broad area, which is often the entire assembly. If the assembly is large, it is often hard to heat the assembly to the flow point of the filler metal as the heat tends to dissipate. Additionally, the relatively high temperature of brazing may damage a small, delicate device and may create a weak area which will break under moderate loads. Like soldering, brazing increases the size of the joint which may end up being non-uniform. Further, a badly brazed joint may look similar to a well-brazed joint and be very low strength. Finally, the joint may not be exposed to temperatures which exceed the melting point of the filler material.

Welding may be used when the assembly is sufficiently robust to withstand the high temperature of the weld process. Welding joins metals by melting and fusing them together, usually with the addition of a welding filler metal. During welding, a concentrated heat at a temperature greater than the melting point of the metals being welded is applied directly to the joint area in order to melt the base metals and the filler metal. The joints produced are usually as strong as or stronger than the metals joined. However, the disadvantages of welding include the high-temperature of the welding process which makes it difficult to apply the weld uniformly over a broad area. Additionally, as in brazing, the high temperature of welding may damage a small, delicate device and may create a weak area near the weld which will break under moderate loads. Further, welding may increase the size of the joint, may require a great deal of skill, and can be expensive and time-consuming.

Adhesive bonding may be used to join two parts together using an adhesive which may be a polymer, plastic or synthetic resin. Adhesive bonding may be used when a device is small and fragile and will be exposed to the blood stream for extended periods of time. An advantage of adhesive bonding is that it generally does not require high temperatures. However, a major shortcoming of adhesive bonding is that it generally does not produce a very high-strength, durable joint, and the bond typically does not hold up at higher temperatures.

Each of these processes compromises one or more important device properties, such that it may decrease device strength, degrade the material, decrease the springiness or flexibility of the material, increase the size of the manufactured device, lessen the smoothness of the material, or decrease the uniformity or precision of the results of the processes, and as a result yield less than ideal results. Additionally, these processes can require heating, and/or be time consuming and thereby increase manufacturing costs.

The present invention utilizes magnetic pulse welding to alleviate problems associated with the prior art. Before the present invention, the applicability of magnetic pulse welding for use in medical devices such as stents had not been demonstrated.

Magnetic Pulse Welding (MPW) is a process that uses high intensity magnetic fields to force assemblies together to form a bond without the heat needed for conventional welds. The assemblies which are forced together may be tubular, but may also comprise other shapes. Some versions of the MPW process are described in U.S. Pat. No. 3,520,049 to Lysenko and U.S. Pat. No. 6,548,791 to Kiterski which are hereby incorporated by reference. The MPW process has been previously utilized in other industries such as the automotive industry to join parts, but its applicability to medical devices to solve the problems of the prior art was previously unknown.

In FIG. 8, in one embodiment, a magnetic pulse welding device 10 stores electrical energy within a bank of capacitors (not shown) and releases the energy through one or more inductor coils 20. Inner and outer components, 30 and 32 respectively, are placed in the vicinity of the inductor coil 20 so that the components are within a magnetic field to be created by the inductor coil 20. The outer component 32 must be conductive, and is generally metallic. However, other conductive materials such as conductive polymers may be used for the outer component 32. The inner component 30 may be conductive or non-conductive, and may be made of a wide variety of material including, but not limited to, any metal or plastic. Generally, the inner and outer components are tubular, but other shapes may be used. The energy released through the inductor coil 20 generates a magnetic field 25 which is strong enough to collapse the conductive outer component 32 inwardly into engagement with the inner component 30. If both of the inner and outer components are metallic, with sufficiently high energy, the inward collapsing velocity will cause the metal of the outer component to penetrate the metal of the inner component forming a metallurgical bond between the components in a process referred to as “cold stage welding.” The metal, or other material, is locally heated to no more than about 30 degrees Celsius. Therefore, no heat-affected zone is created, and the metal, or other material, is not degraded. The weld becomes the strongest part of the assembly. The high collapsing velocity pushes metal, or other material, well beyond its yield strength and into its plastic region, resulting in permanent deformation with no springback. Metals or other materials with lower conductivity can also be processed.

The MPW process takes less than 100 microseconds. No gases, fillers, fluxes, or other materials are needed to achieve the weld. Additionally, a gap is preferred between the parts for the process to work most effectively, so tight tolerances are not critical. MPW works as long as one or more of the components is conductive. The more conductive the part, the less energy is required to achieve a weld. Metals that easily weld are aluminum and copper. However, MPW has been successful in welding a number of similar and dissimilar metals. Some examples include: aluminum to aluminum; aluminum to copper; aluminum to magnesium; aluminum to titanium; copper to copper; copper to steel; copper to brass; nickel to titanium; nickel to nickel; and steel to steel.

Magnetic pulse welding can also be used for joining or crimping parts that do not necessarily need a metallurgical bond, such as a metal to a nonmetallic part. It can create a mechanical lock on ceramics, polymers, rubber, and composites. As a result, adhesives, sealants, and mechanical crimps are not necessary to join the components. In one embodiment, this may be accomplished by essentially shrink-wrapping (or fitting) a conductive component over a non-conductive component through the use of a high energy magnetic field generated by an inductor positioned near the conductive component. In another embodiment, this may be accomplished by expanding an inner conductive component into an outer non-conductive component through the use of a high energy magnetic field generated by an inductor within the inner conductive component. In other embodiments, the inductor may be in the vicinity of the components and may generate a magnetic field to expand the inner conductive component into an outer non-conductive component.

In FIG. 9 the inductor coil 20 is positioned within a conductive inner component 30 which is disposed within an outer component 32 which may be conductive or non-conductive. The outer and inner components are generally tubular, but may be any shape. The inner component is forced to move away from the coil at a high speed due to the magnetic force created by the coil and subsequently strikes the material of the outer component at a high velocity to form a bond between the components utilizing an expansion fit. The inner component may be any material which is conductive such as metal or a conductive polymer. The outer component may be made of a wide variety of conductive or non-conductive materials such as metals, polymers, or plastic.

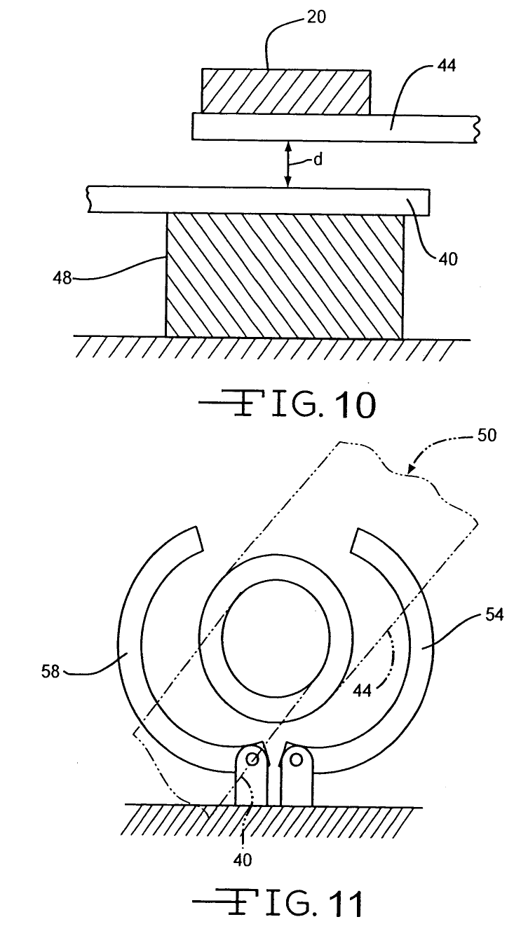

FIG. 10 shows another embodiment in which a conductive component 40 is placed at a distance d from another component 44 which may be conductive or non-conductive. The conductive component may be any type of conducting material, including but not limited to any type of metal or conductive polymer. The component 40 is restrained from movement using a clamp 48 or other type of restraining device. The inductor coil 20 is positioned above the component 44. The component 44 is forced to move away from the inductor coil 20 toward the component 40 at a high velocity due to the magnetic force emulating from the coil. If the magnetic field is of sufficiently high energy, the components 40 and 44 will be welded together in a permanent bond when the component 44 strikes the component 40. The arrangement of FIG. 10 makes it possible to use the MPW process when the components 40 and 44 will form a closed loop thereby allowing the inductor coil 20 to be removed from the arrangement.

As shown in FIG. 11, in another embodiment a split inductor coil 50 may be utilized to apply the MPW process when the components 40 and 44 will form a closed loop, to allow the split inductor coil 50 to be removed from the arrangement. The split inductor coil 50 may be comprised of two or more parts 54 and 58 in varying arrangements which may be pulled apart and then joined together around a closed loop system 40 and 44 to allow the MPW process to be applied. After the MPW process is completed, the parts 54 and 58 may be pulled apart and the closed loop system 40 and 44 may be removed. In the arrangement shown, the outer component 44 must be conductive and the inner component 40 may be any type of non-conductive or conductive material. However, in other embodiments, varying arrangements for the components may be used inside a split inductor such as an arrangement where two components, at least one of which is conductive, are placed parallel to one another inside the split inductor.

The MPW process makes the joining of metallic assemblies, or assemblies using one metallic material and a non-metallic material, possible with the strength of a weld but without the resulting annealing or damage to the parent material. Although previously unknown in the prior art of the medical field, utilizing MPW to manufacture a medical device makes it possible to make small, fragile medical devices, such as a stent, that can have welds without weak or soft areas. In the following embodiments, whenever the MPW process is referred to, the above MPW disclosure is applicable for purposes of forming the MPW weld in each embodiment.

FIG. 12 shows a preferred embodiment of utilizing MPW to join components in a stent. An end e of a stent connecting member 5 is inserted into an aperture a running radially through a portion of a conductive stent arm section 6 so that the end e is disposed within the arm section 6. The arrangement is positioned within the area A between two open parts 54 and 58 of a split inductor 50. The two parts 54 and 58 of the split inductor 50 are then pushed together to complete the inductor 50 and the MPW process is applied to collapse the arm section 6 around the end e of the connecting member 5 to permanently join the components. The arm section 6 may be conductive, and the connecting member 5 may be conductive or non-conductive. After the connecting member 5 and the arm section 6 have been joined together by the MPW process, the two parts 54 and 58 of the split inductor 50 are pulled apart to remove the inductor 50 from the arrangement. Although a split inductor is used in a preferred embodiment, other types of inductor arrangements may be used to apply MPW. For example, the inductor arrangement of FIG. 10 may be utilized.

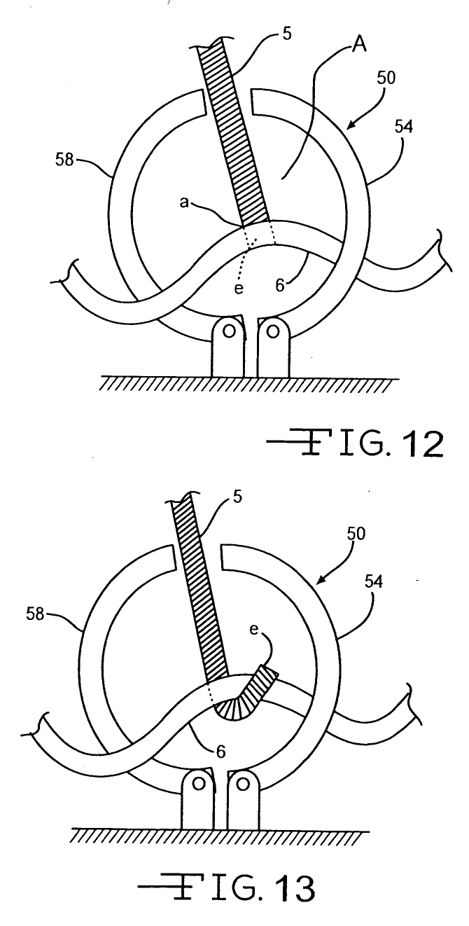

Other embodiments may utilize additional methods of positioning the connecting member with the associated arm section. For example, in FIG. 13 an end e of a stent connecting member 5 is wound around a portion of a stent connecting arm section 6. The stent connecting member 6 may be conductive and the arm section 6 may be conductive or non-conductive. The arrangement is disposed within the parts 54 and 58 of a split inductor 50. The parts 54 and 58 are closed and the MPW process is applied. After the components have been joined by the MPW process, the parts 54 and 58 are pulled apart to remove the inductor 50. In other embodiments, the inductor may be in the vicinity of the components and may generate a magnetic field to join the components.

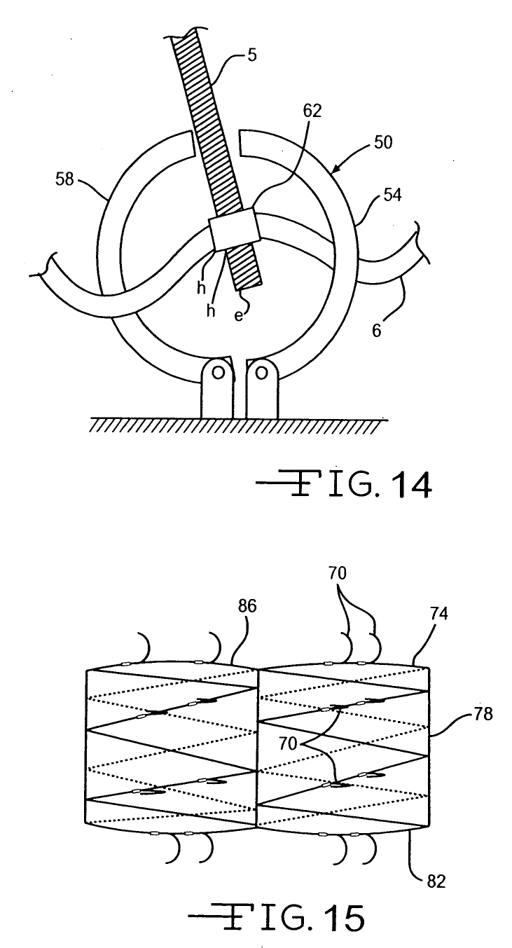

As shown in FIG. 14, in another embodiment an end e of a stent connecting member 5 may be extended through an axially extending hole h in a cylindrical, hollow, conductive crimping member 62. Similarly, a stent connecting arm section 6 may be extended through another radially extending hole h in the hollow crimping member so that portions of both the stent connecting member 5 and the stent connecting arm section 6 are disposed within the hollow crimping member 62. The crimping member 62 may be other shapes in other embodiments, but must be conductive. The stent connecting member 5 and arm section 6 may be conductive or non-conductive. The arrangement is disposed within the parts 54 and 58 of a split inductor 50. The parts 54 and 58 are closed and the MPW process is applied. The force of the magnetic field generated by the inductor 50 collapses the conductive crimping member 62 around the connecting member 5 and the arm section 6. After the components have been joined by the MPW process, the parts 54 and 58 are pulled apart to remove the inductor 50.

These examples are not exhaustive and other methods of positioning the members in alignment for MPW welding are contemplated and included in the present invention.

In another embodiment, the present invention is utilized in assembling an anchoring barb to a Z-stent using the process of MPW to eliminate the problems associated with the prior art. To address the problem of device migration, as shown in FIG. 15, stent graft manufacturers sometimes place a series of barbs or hooks 70 that extend outward from the main body 74 of the prosthesis device 78, typically at its proximal end 82, either by attaching the barbs to the stent frame 86 with solder or by some other bonding technique, or to the graft material, typically by suturing. It has been observed that sutures attaching barb stents to the graft material are subject to breakage due in part to the flexibility of the graft material and the considerable pulsatile forces of arterial blood acting on the device. These forces have been known to directly contribute to the detachment between the graft portion and anchoring stent.

It has also been observed that barbs soldered or otherwise attached to the stent frame by conventional methods are subject to fracture, detachment, or other failure, especially when the forces become concentrated at a particular location along the stent graft. Unfortunately, simply making the barbs stronger to prevent fracture can result in damage to the anchoring tissue. Furthermore, adding rigidity to any outward-projecting barbs may compromise the ability of the device to be compressed and loaded into a delivery system. The use of multiple barbs can prevent catastrophic migration of the device. Yet, while a single barb failure should not result in the migration of the device and may not represent a problem clinically, a barb fracture or failure is nevertheless currently classified as an adverse event that manufacturers seek to avoid.

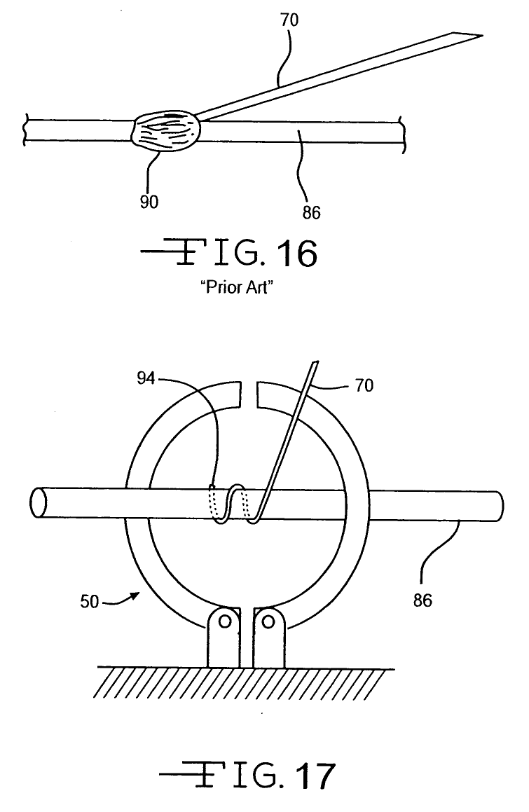

One prior solution to address barb failure was disclosed in U.S. Pat. No. 5,720,776 to Chuter et al., depicted in FIG. 16. The barb 70 includes both the traditional solder bond 90 and a mechanical attachment (not shown) below the bond 90 to attach the barb 70 to the stent frame 86. In addition, the barb is made laterally flexible to help accommodate forces acting at the anchor point. These improvements help ensure that the barb does not readily detach from the stent due to a failure of the solder joint alone. While the combination of both solder and a mechanical means to affix the barb to the stent has proved effective in most respects, this area of the barb remains subject to stresses, such as from cyclic loading resulting from the pulsatile action of the vessel. What is needed is a barb-to-stent connection that is better able to accommodate bending and shear stresses in order to further reduce the likelihood of barb failure due to the fracture of the connection.

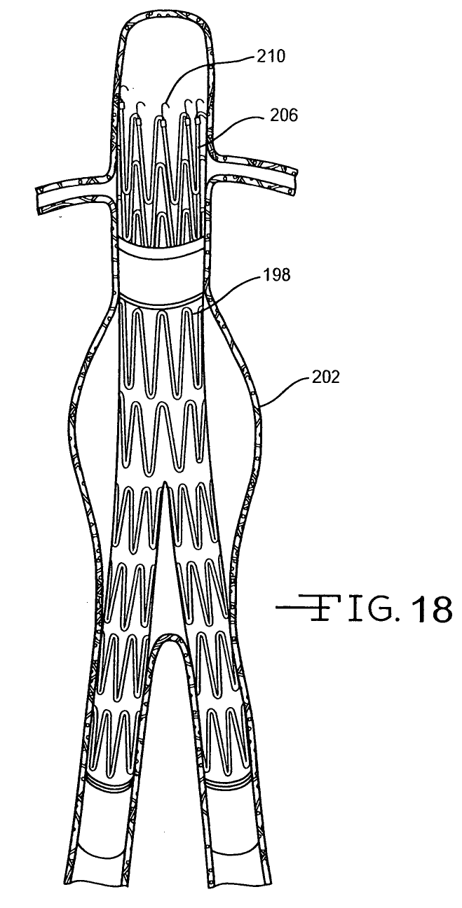

Previously unknown in the art of medical devices, MPW resolves these problems. FIG. 17 shows an embodiment utilizing MPW in a barb to stent connection. An end 94 of the barb 70 is helically wound around a portion of a stent frame 86 and then the assembly is positioned inside a split inductor coil 50. The assembly is welded using MPW. The resulting connection retains the strength of the connection and avoids having a solder or adhesive that will eventually corrode or break-down after prolonged exposure to body fluids. Further, other connection arrangements applying MPW are contemplated such as the arrangements previously discussed.

In yet another embodiment, as shown in FIG. 18, the present invention is utilized in a bifurcated stent device 198 with a Y-shaped structure which is used to treat an abdominal aorta aneurysm 202 (AAA device). Such an AAA device is disclosed in PCT international publication number WO 98/53761 to William A. Cook which is hereby incorporated by reference. To eliminate the problems associated with the prior art, the MPW process as previously disclosed is utilized to join the anchoring barbs 210 to the end of the stent 206.

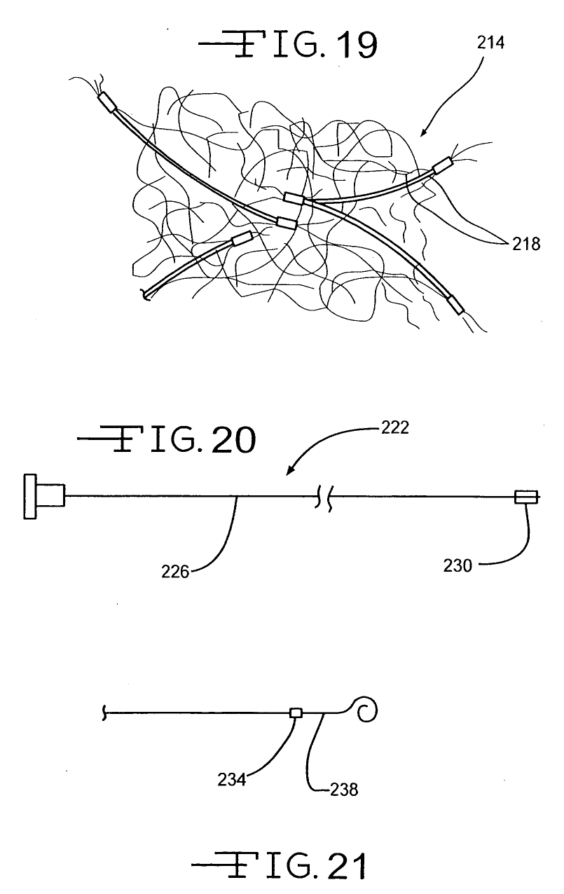

In another embodiment, as shown in FIG. 19, MPW is utilized in a clot filter 214 to eliminate the problems associated with the prior art. A clot filter can be placed in a patient's blood vessel, percutaneously, when needed and removed, percutaneously, when the need for such a filter has passed. The filter wires 218 are typically secured together by laser welding, brazing, or crimping. However, these processes may damage the material and create weak areas where the wires could break under moderate loads. The use of MPW to form the connections between the filter wires solves these problems.

Additionally, in another embodiment as shown in FIG. 20, MPW is well suited to attach rods to cylinder type assemblies. A stiffening cannula 222 with a rod 226 and a collar 230 at the distal end is used to engage the interior of the tip of a catheter so that it can be pushed through a puncture site and into the patient. The soldering method currently used to attach the collar 230 to the rod 226 is a slow, time consuming process which delivers marginal strength. MPW eliminates these problems.

In addition to assembling metal components, as shown in FIG. 21 MPW can be used to “shrink” or force small metal bands 234 into a tight, interference fit over catheters 238 for radiopaque marker bands. Currently, these devices are made by first stretching the catheter so as to reduce its outer diameter, sliding the bands on the tubing, then allowing the tubing to return to its original outer diameter. The result is that the bands are tight and fairly well lodged on the catheter. However, as the catheter expands back to or near its original outer diameter, the material also shortens. This makes accurate placement of the bands somewhat difficult. In addition, the “tightness” or security of the marker bands on the tubing is only marginally adequate to keep the markers in place on the catheter during use. The MPW process would allow the bands to be “shrunk” onto the tube at the precise location and spacing and allow the band to be as tight a fit as needed to ensure that the band will not move or come off the catheter during use. Similarly, the MPW process could be used to place marker bands on other types of medical devices, such as placing a gold marker band on a stent.

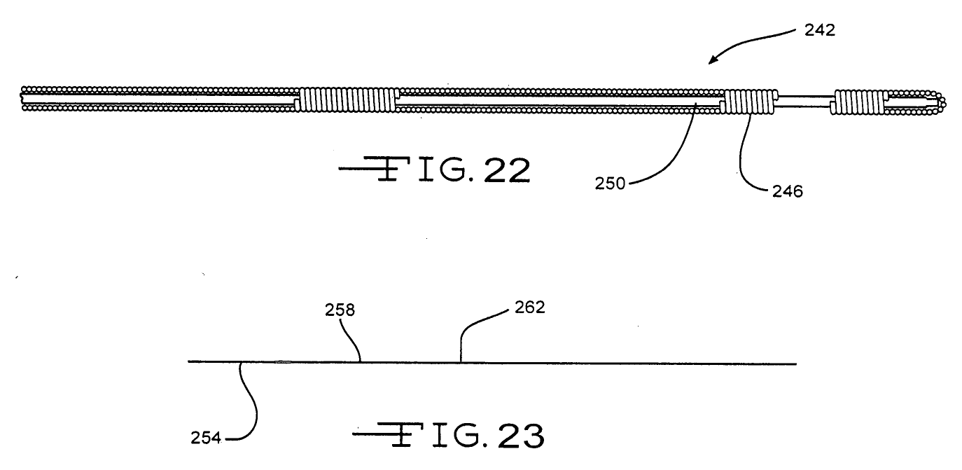

In another embodiment for wire guide assemblies, as shown in FIG. 22, the joints of wire guide assemblies 242 are typically coil 246 to mandrel 250 connections. Solder is usually used in these assemblies so that the mandrel is not softened or annealed at the joint. If the mandrel has a soft or annealed area on its length, the wire will bend during use, adversely affecting its ability to be manipulated or guided through the anatomy. MPW would allow the joint to be made as strong as a weld without the resulting “kink point” caused by the annealing heat of arc or TIG welding.

As shown in FIG. 23, the MPW process also can be used in a tip deflecting wire 262 for the assembly of a needle cannula 254 to the wire 258, and in other devices where cannula is assembled or attached to wire guide coils. Further, as shown in FIG. 24, the MPW process can be used for the assembly of a needle cannula 266 to a metal hub 270.

In the case of retrieval baskets 274 as shown in FIG. 25, the wires 278 of the basket are usually joined to the shaft 282 of the device with solder. Again, this is done so as to not soften the wires of the basket. The basket wires are formed into an “open” or round shape so that it can capture and hold stones or other foreign bodies for removal from a body lumen. The basket is delivered in a collapsed, small diameter shape through the lumen of a catheter. When the basket exits the tip of the catheter, the spring temper of the basket wires cause it to open up into the round shape. If the basket wires are annealed, as would be the case if they were welded together by conventional arc welding, the resulting soft areas would compromise the ability of the basket to spring into the proper open shape. In addition, retrieval baskets are often used to break or crush the stones or calcification they capture before they can be removed percutaneously from the vascular system. The basket is retracted into the delivery catheter and pulled with enough force to cause the collapsing action to break or crush the stone. These forces are often quite high and can cause solder joints to fail. MPW joints would greatly increase the strength of the joint making it possible to apply more force to the basket for breaking the stones.

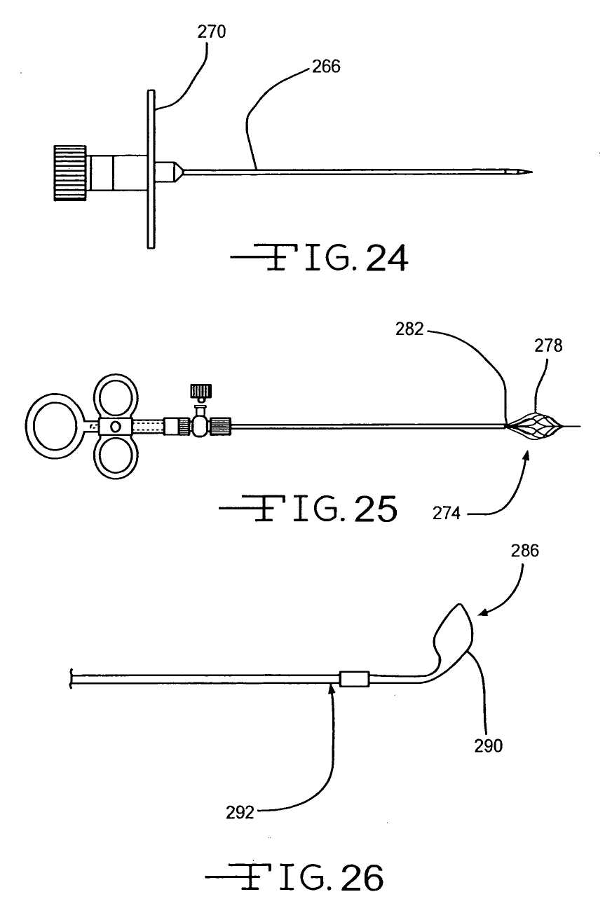

As shown in another embodiment, FIG. 26, a retrieval snare 286 is a device similar to a basket in that it is used for foreign body retrieval from a body lumen. It is typically used to retrieve catheter fragments and broken guide wires. The loop snare 290 must open when extended from the tip of the sheath and be securely attached to the snare shaft so that the fragments can be captured and removed. The assembly of the loop snare 290 connection to the wire 292 would benefit from the MPW in a way similar to the retrieval basket.

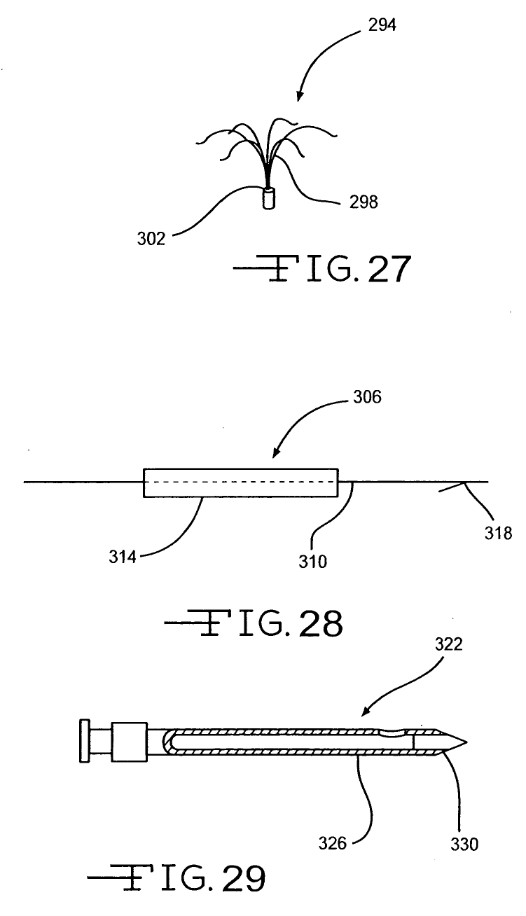

FIG. 27 shows another embodiment for a spider occluder device 294, which is a permanent implant device that is used to prevent the migration of other occlusive devices in the blood stream. The legs or arms 298 of the spider are joined together at one end at a central point 302, and radiate outwardly in multiple directions when deployed so as to engage the vessel wall. The spider occluder device is usually pushed from a short shipping cartridge into a catheter with a stiff wire guide. It is then pushed through the catheter and out the tip into the artery. When the spider exits the tip of the catheter, the legs spring open radially and engage the vessel wall. The spider occluder is assembled with solder at the central joint because the heat needed to weld the wires together would anneal or soften the wires. As a result, the legs would not spring open far enough or with sufficient force to engage the vessel wall. The MPW process would allow a much more secure joining process that would not anneal or change the characteristics of the wire. Also, the solder used to join the wires together may corrode over time. Normally, the spider occluder is fully incorporated into the vessel wall by the time the solder has corroded away and there is no migration. However, a MPW welded assembly would last much longer than a soldered joint, and corrosion and eventual joint failure would no longer be a concern.

FIG. 28 shows a breast lesion localization device 306 which is used by a radiologist to guide a surgeon during surgery directly to the lesion. This reduces the amount of time and surgical exploration needed to find and remove a lesion from the breast. The localizer wire 310 is introduced through a needle that is placed by the radiologist under fluoroscopic guidance. Once the needle point is in the lesion, the radiologist advances the localizer wire through the needle until the hook at the distal end of the wire exits the tip of the needle and anchors in the lesion. The needle is then removed, leaving the localizer wire behind, protruding from the patient's skin. The surgeon surgically follows the wire into the breast to the hook in the lesion. The lesion and surrounding tissue are then removed. As the surgeon cuts through the breast tissue along the wire, he or she needs to know where the end of the wire and the lesion. Depth marks along the wire are hard to see; therefore, a cannula sleeve 314 is attached just proximal to the hook 318 so that the surgeon will know the lesion is closed when the cannula sleeve 314 is encountered. This cannula marker sleeve is usually attached to the wire with solder. Solder is used in this application because it is important not to anneal or soften the wire. A bent localizer wire is very difficult for the surgeon to follow accurately. Also, the assembly must be smooth and low profile so as to allow the wire to pass through the smallest possible gauge needle. The use of MPW would allow a secure joint without high heat and actually reduce the diameter at the joints by the compression of the cannula marker over the wire during the MPW process rather than add to it as is usually the case with welding and soldering.

Another application for MPW is shown in FIG. 29 which shows a hollow needle 322 used for abscess drainage, fluid delivery and flushing. These needles are usually fairly large in diameter (10 to 6 gauge), have a sharp conical point and a large side hole that communicates with the lumen of the needle just proximal to the point. Currently, these needles are made by brazing a short rod 326 into the distal end of the needle point 330. The point is then ground across the joint so that the needle cannula blends into the conical point on the rod. Brazing is used because it produces a relatively strong joint and the material can be “sweat” or flowed into the joint all along the contact area between the rod and needle cannula. By making the joint all along the contact length, the needle cannula and rod can be ground together to form the point without worry of grinding away the joining material, leaving the joint weak or compromised. Flowing the solder or brazing material into this joint requires a high level of brazing skill and it is extremely difficult to visually or non-destructively assure that the brazing material has flowed evenly through the joint. The brazed assemblies have a ring of softer brazing material around the conical point where the two parts are joined. Since the brazing material is softer, it grinds at a different rate than the stainless steel of the needle cannula and the tip rod. This results in a bump or catch that interferes with the passage of the needle through tough, fibrous tissue. The use of MPW would allow the rod and needle cannula to reliably be joined all along the contact length by collapsing the needle cannula over the rod. In addition, since the needle cannula and rod would be welded together, the transition from the needle cannula to the rod on the conical point would be very smooth and unnoticeable to the user.

These examples show that there is a myriad of applications for the invention in medical device assemblies. Anywhere high strength with low or no heat is needed, MPW can be applied to join components. Even dissimilar metals, such as stainless steel to nitinol, as used in several wire guide designs, can benefit from MPW. The demanding requirements of many medical device assemblies for maximum strength, corrosion resistance, and preservation of parent material characteristics make MPW uniquely well suited to a vast number of medical device applications.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that the appended claims, including all equivalents thereof, are intended to define the scope of the invention.

1. A medical device for use in or on a mammalian body comprising:
a first component;
a second component;
and a connection formed between said first component and said second component, wherein said connection is formed by magnetic pulse welding.

2. The medical device of claim 1 wherein the medical device is one of a stent, a clot filter, a stiffening cannula, a catheter, a wire guide assembly, a needle cannula assembly, a retrieval basket assembly, a retrieval snare device, a spider occluder assembly, a breast lesion localization needle depth marker assembly, a needle point assembly, and a marker assembly.

3. The medical device of claim 1 wherein the first and second components are metallic, and at least a portion of said first component and at least a portion of said second component are joined in a metallurgical bond without degradation of the metal of either component.

4. The medical device of claim 1 wherein the connection is formed by disposing at least a portion of said first component within at least a portion of said second component with both of said components being disposed within an inductor.

5. The medical device of claim 1 wherein the connection is formed by disposing at least a portion of said first component within at least a portion of said second component and an inductor is positioned within said portion of said first component.

6. The medical device of claim 1 wherein at least a portion of said first component and at least a portion of said second component are substantially aligned and an inductor is disposed on a side of one of said first and second components.

7. The medical device of claim 1 wherein said first and second components are metallic.

8. The medical device of claim 1 wherein said first component is metallic and said second component is non-metallic.

9. The medical device of claim 1 wherein the magnetic pulse welding takes less than 100 microseconds.

10. The medical device of claim 1 wherein the first component and the second component are heated to less than about thirty degrees Celsius during the magnetic pulse welding.

11. The medical device of claim 1 wherein said first and second components are tubular.

12. A medical device for use in or on a mammalian body comprising:
a first conductive, metallic component; and
a second conductive, metallic component, wherein at least a portion of said first component and at least a portion of said second component are joined in a metallurgical bond without degradation of the metal of either component.

13. The medical device of claim 12 wherein the medical device is one of a stent, a clot filter, a stiffening cannula, a catheter, a wire guide assembly, a needle cannula assembly, a retrieval basket assembly, a retrieval snare device, a spider occluder assembly, a breast lesion localization needle depth marker assembly, a needle point assembly, and a marker assembly.

14. The medical device of claim 12 wherein at least a portion of said first component and at least a portion of said second component are joined in a metallurgical bond without substantial degradation of the metal of either component through magnetic pulse welding.

15. The medical device of claim 14 wherein the magnetic pulse welding comprises disposing at least a portion of said first component within at least a portion of said second component with both of said components being disposed within an inductor.

16. The medical device of claim 14 wherein the magnetic pulse welding comprises disposing at least a portion of said first component within at least a portion of said second component and positioning an inductor within said portion of said first component.

17. The medical device of claim 14 wherein at least a portion of said first component and at least a portion of said second component are substantially aligned and an inductor is disposed on a side of one of said first and second components.

18. The medical device of claim 14 wherein the magnetic pulse welding takes less than 100 microseconds.

19. The medical device of claim 14 wherein the first component and the second component are heated to less than about thirty degrees Celsius during the magnetic pulse welding.

20. The medical device of claim 12 wherein said first and second components are tubular.

21. A method of assembling at least a portion of a medical device for use in or on a mammalian body comprising:
providing a first component, a second component, and an inductor, wherein at least one of said first and second components is conductive;
positioning at least a portion of said first component near at least a portion of said second component and in the vicinity of said inductor; and
supplying a current pulse to the inductor to generate a magnetic field, thereby causing at least a portion of said first component to form a connection with at least a portion of said second component by magnetic pulse welding.

22. The medical device of claim 21 wherein the medical device is one of a stent, a clot filter, a stiffening cannula, a catheter, a wire guide assembly, a needle cannula assembly, a retrieval basket assembly, a retrieval snare device, a spider occluder assembly, a breast lesion localization needle depth marker assembly, a needle point assembly, and a marker assembly.

23. The medical device of claim 21 wherein the first and second components are metallic, and said welded portion of said first component and said welded portion of said second component are joined in a metallurgical bond without substantial degradation of the metal of either component.

24. The medical device of claim 21 wherein at least a portion of said first component is positioned within at least a portion of said second component and said second component is positioned inside of said inductor.

25. The medical device of claim 21 wherein at least a portion of said first component is positioned within at least a portion of said second component and said inductor is positioned inside of said first component.

26. The medical device of claim 21 wherein at least a portion of said first component and at least a portion of said second component are substantially aligned and said inductor is disposed on a side of one of said first and second components.

27. The medical device of claim 21 wherein said first and second components are metallic.

28. The medical device of claim 21 wherein said first component is metallic and said second component is non-metallic.

29. The medical device of claim 21 wherein the magnetic pulse welding takes less than 100 microseconds.

30. The medical device of claim 21 wherein the first component and the second component are heated to thirty degrees Celsius or less during the magnetic pulse welding.

31. The medical device of claim 21 wherein said first and second components are tubular.

32. A method of assembling at least a portion of a medical device for use in or on a mammalian body comprising:
providing a first component, a second component, and an inductor, wherein at least one of said first and second components is conductive;
positioning at least a portion of said first component near at least a portion of said second component and in the vicinity of said inductor; and
explosively compelling one of said first and second components towards the other of said components through the use of a magnetic field so as to form a connection between the first and second components.

33. A method of assembling at least a portion of a medical device for use in or on a mammalian body comprising:
providing a first component made of a material, a second component made of a material, and an inductor, wherein at least one of said first and second components is conductive;
positioning at least a portion of said first component near at least a portion of said second component and in the vicinity of said inductor; and
compelling one of said first and second components towards the other of said components through the application of a magnetic field created by said inductor, without substantially heating or degrading the material of either of said first and second components.
